(19) United States
(12) Patent Application Publication
(10) Pub. No.: US 2018/0093888 A1
(43) Pub. Date: Apr. 5, 2018

(54) METHODS FOR CONVERSION OF CO2 INTO SYNGAS

(71) Applicants: Aghaddin MAMEDOV, Sugar Land, TX (US); Shahid SHAIKH, Sugar Land, TX (US); Clark REA, Sugar Land, TX (US); SABIC Global Technologies B.V., Bergen op Zoom (NL)

(72) Inventors: Aghaddin MAMEDOV, Sugar Land, TX (US); Shahid SHAIKH, Sugar Land, TX (US); Clark REA, Sugar Land, TX (US)

(21) Appl. No.: 15/566,978

(22) PCT Filed: Apr. 21, 2016
(86) PCT No.: PCT/US2016/028594

§ 371 Date: Oct. 16, 2017

Related U.S. Application Data
(60) Provisional application No. 62/154,308, filed on Apr. 29, 2015.

(51) Int. Cl. C01B 3/40; C07C 1/12; B01J 23/889; B01J 35/00; C10G 2/00

(52) U.S. Cl. CPC C01B 3/40; C07C 1/12; B01J 23/8892; B01J 35/002; C10G 2/50; C07C 2523/889; C01B 2203/062; C01B 2203/04; C01B 2203/02; C07C 2523/34; C07C 2523/72; C10G 2/30

(57) ABSTRACT

Methods of preparing syngas are provided. An exemplary method can include hydrogenation of carbon dioxide (CO₂) via a reverse water gas shift (RWGS) reaction. Catalysts that include Cu and/or Mn can be used, and the RWGS reaction can be conducted at a temperature greater than 600° C. The syngas produced from hydrogenation of CO₂ can be used to generate light olefins via a Fischer-Tropsch synthesis (FT) reaction.

### CROSS-REFERENCE TO RELATED APPLICATIONS

This application claims the benefit of priority of U.S. Provisional Patent Application No. 62/154,308, filed Apr. 29, 2015, which is hereby incorporated by reference in its entirety.

### FIELD

The presently disclosed subject matter relates to methods and systems for conversion of carbon dioxide (CO₂) into synthesis gas (syngas) via hydrogenation of CO₂. The presently disclosed subject matter also relates to methods and systems for preparation of light olefins.

### BACKGROUND

Light olefins (e.g., C₂-C₄ olefins) are important industrial chemicals. Light olefins such as ethylene, propylene, and butene isomers (1-butene, cis-2-butene, trans-2-butene, and isobutylene) are widely used as feedstocks for polymerization, among many other uses.

There is interest in preparing light olefins and other chemical feedstocks from carbon dioxide (CO₂). CO₂ is an abundant and economical starting material. Use of CO₂ as a feedstock in chemical processes can reduce emissions of CO₂ and improve overall sustainability.

Synthesis gas (also known as syngas) is a mixture of carbon monoxide (CO) and hydrogen (H₂). Syngas can optionally contain other components, including CO₂, water (H₂O), methane (CH₄), and/or nitrogen (N₂). Syngas can be prepared from CO₂ by reaction of CO₂ with H₂. This process can be described as a hydrogenation of CO₂. CO₂ and H₂ can react to form carbon monoxide (CO) and water (H₂O) through a reverse water gas shift (RWGS) reaction. The RWGS reaction is endothermic and can be described by Equation 1:

CO₂+H₂→CO+H₂O Δ_{R}H⁰_{300° C.}=38 kJ/mol (1)

The RWGS reaction is reversible; the reverse reaction (from CO and H₂O to CO₂ and H₂) is known as the water gas shift reaction. The RWGS reaction can be conducted under conditions that provide partial conversion of CO₂ and H₂, thereby creating an overall product mixture that includes CO₂, H₂, CO, and H₂O. CO₂ and H₂O can optionally be removed from such a product mixture, thereby providing a purified syngas mixture containing primarily CO and H₂.

Syngas is a versatile mixture that can be used to prepare light olefins, methanol, and many other important industrial chemicals. For example, syngas can undergo a Fischer-Tropsch synthesis (FT) reaction to provide a mixture of hydrocarbons that includes light olefins. The FT reaction is exothermic and can be described by Equation 2:

CO+2H₂→“CH₂”+H₂O Δ_{R}H⁰_{300° C.}=−166 kJ/mol (2)

In Equation 2, “CH₂” represents a generic hydrocarbon moiety that can be incorporated into a larger molecule, e.g., ethylene (C₂H₄) or propylene (C₃H₆).

Thus, as presented above, light olefins can be generated from CO₂ by first converting CO₂ to syngas through a RWGS reaction and subsequently converting syngas to light olefins through a FT reaction. The efficiency of preparation of light olefins from syngas can depend on the composition of the syngas. As indicated in Equation 2, syngas containing H₂ and CO in a molar ratio (H₂:CO) of about 2:1 can be useful for the FT reaction. A disadvantage of many existing methods of preparing syngas is that they tend to produce syngas having a H₂:CO molar ratio of 3:1 or greater. For example, steam reforming of methane tends to generate syngas with a H₂:CO molar ratio of 3:1 or greater.

An issue encountered with preparation of syngas via reduction of CO₂ through the RWGS can be poor conversion. As noted above, the RWGS reaction is reversible and endothermic. Increasing reaction temperature can increase conversion of CO₂ and H₂ to CO and H₂O, but increasing the temperature of RWGS reactions is also known to increase side reactions. For example, U.S. Patent Application Pub. No. 2013/0150466 notes that conducting a RWGS reaction at too high of a reaction temperature can induce unwanted reactions. Additional problems encountered during preparation of syngas via reduction of CO₂ through the RWGS reaction can include poor catalyst stability and poor yield.

Thus, there remains a need in the art for new methods for conversion of CO₂ into syngas with improved H₂:CO ratios, reduced side reactions, improved catalyst stability, improved yield, and improved overall economy.

### SUMMARY OF THE DISCLOSED SUBJECT MATTER

The presently disclosed subject matter provides methods of preparing syngas as well as methods of preparing light olefins.

In one embodiment, an exemplary method of preparing syngas can include providing a reaction chamber. The reaction chamber can include a solid-supported catalyst. The solid-supported catalyst can include copper (Cu) and manganese (Mn). The method can further include feeding a reaction mixture that includes H₂ and CO₂ to the reaction chamber. The method can additionally include contacting H₂ and CO₂ with the catalyst at a reaction temperature greater than 600° C., to provide a product mixture that includes H₂ and CO.

In certain embodiments, the catalyst can include Cu and Mn in a molar ratio (Cu:Mn) of about 4:1 to about 1:4. The catalyst can include Cu and Mn in a molar ratio (Cu:Mn) of about 1:1. In certain embodiments, the catalyst can include one or more solid supports selected from the group consisting of alumina (Al₂O₃), magnesia (MgO), silica (SiO₂), titania (TiO₂), and zirconia (ZrO₂). In certain embodiments, the catalyst can include one or more additional metals selected from the group consisting of lanthanum (La), calcium (Ca), potassium (K), tungsten (W), and aluminum (Al). In certain embodiments, the catalyst can include about 10% Cu and about 10% Mn, by weight. The remainder of the catalyst can be oxygen (i.e., the oxygen present in a metal oxide) and solid support (e.g., Al₂O₃).

In certain embodiments, the catalyst can be free of chromium (Cr). That is, the catalyst can be a catalyst that does not include Cr. In certain embodiments, the catalyst can include less than about 1% Cr, by weight. The catalyst can include less than about 0.1% Cr, by weight. The catalyst can include less than about 0.01% Cr, by weight.

In certain embodiments, the reaction mixture can include H₂ and CO₂ in a molar ratio (H₂:CO₂) of about 1.6:1.

In certain embodiments, the reaction temperature can be greater than about 625° C. The reaction temperature can be greater than about 650° C. The reaction temperature can be about 670° C.

In certain embodiments, the product mixture can include H₂ and CO in a molar ratio (H₂:CO) of about 1:1 to about 3:1. The product mixture can include H₂ and CO in a molar ratio (H₂:CO) of about 1.5:1 to about 3:1. The product mixture can include H₂ and CO in a molar ratio (H₂:CO) of about 2:1 to about 3:1. The product mixture can include H₂ and CO in a molar ratio (H₂:CO) of about 2.5:1.

In certain embodiments, the product mixture can further include CO₂ and H₂O. The product mixture can include less than about 25% CO₂, by mole. The product mixture can include less than about 20% CO₂, by mole. In certain embodiments, the method can further include separating at least a portion of CO₂ and H₂O from the product mixture to provide purified syngas.

In one embodiment, an exemplary method of preparing light olefins can include providing a reaction chamber. The reaction chamber can include a solid-supported catalyst. The catalyst can include Cu and Mn. The method can further include feeding a reaction mixture that includes H₂ and CO₂ to the reaction chamber and contacting H₂ and CO₂ with the catalyst at a reaction temperature greater than 600° C. to provide a product mixture that includes H₂, CO, CO₂, and H₂O. The method can additionally include separating at least a portion of CO₂ and H₂O from the product mixture to provide purified syngas. The method can further include subjecting purified syngas to a Fischer-Tropsch synthesis (FT) reaction to provide light olefins.

Conversion of syngas to olefins by the FT reaction may include catalysts known in the art, including but not limited to Fe—Mn/Al₂O₃, Fe—Mn/SiO₂, Co—Mn/Al₂O₃, and Co—Mn/SiO₂ using reaction conditions specified for this reaction, including, for example, temperatures in the range of from about 240° C. to about 400° C., and pressure values in the range of from about 20 bar to about 50 bar.

In one embodiment, CO₂ usually produced as a byproduct in preparation of olefins from syngas can be recycled back to into hydrogenation of CO₂, increasing overall selectivity for hydrocarbons and overall carbon efficiency in the preparation of light olefins from CO₂.

### BRIEF DESCRIPTION OF THE DRAWINGS

FIG. 1 is a schematic diagram presenting an exemplary process for preparation of hydrocarbons (e.g., light olefins) from CO₂. CO₂ can be recycled through the process. FIG. 1 depicts the integration of RWGS and FT reactions with recycling of the FT byproduct CO₂.

### DETAILED DESCRIPTION

There remains a need in the art for new methods of preparing syngas from CO₂. The presently disclosed subject matter provides methods of converting CO₂ and H₂ into syngas with improved H₂:CO ratios, reduced side reactions, improved catalyst stability, and improved yield. The presently disclosed subject matter also provides improved methods of preparing light olefins. The presently disclosed subject matter includes the surprising discovery that solid-supported catalysts containing Cu and Mn can be used to promote hydrogenation of CO₂ at temperatures greater than 600° C., greater than 625° C., and greater than 650° C. Such catalysts can be stable at these high temperatures, and the use of reaction temperatures greater than 600° C. can provide improved conversion of CO₂, improved ratios of H₂:CO, and improved yield. Moreover, the catalysts can be free of Cr or contain low levels of Cr (e.g., less than about 1%).

As used herein, the term “about” or “approximately” means within an acceptable error range for the particular value as determined by one of ordinary skill in the art, which will depend in part on how the value is measured or determined, i.e., the limitations of the measurement system. For example, “about” can mean a range of up to 20%, up to 10%, up to 5%, and or up to 1% of a given value.

### Reactors and Reaction Chambers

The methods of the present disclosure can involve fixed bed isothermal or adiabatic reactors suitable for reactions of gaseous reactants and reagents catalyzed by solid catalysts. The reactor can be constructed of any suitable materials capable of holding high temperature, for example from about 600° C. to about 780° C. Non-limiting examples of such materials can include metals, alloys (including steel), glasses, ceramics or glass lined metals, and coated metals. The reactor can also include a reaction vessel enclosing a reaction chamber.

The dimensions of the reaction vessel and reaction chamber are variable and can depend on the production capacity, feed volume, and catalyst. The geometries of the reactor can be adjustable in various ways known to one of ordinary skill in the art.

In certain embodiments, reaction conditions within the reaction chamber can be isothermal. That is, hydrogenation of CO₂ can be conducted under isothermal conditions. In certain alternative embodiments, a temperature gradient can be established within the reaction chamber. For example, hydrogenation of CO₂ can be conducted across a temperature gradient using an adiabatic reactor.

The pressure within the reaction chamber can be varied, as is known in the art. In certain embodiments, the pressure within the reaction chamber can be atmospheric pressure, i.e., about 1 bar.

### Catalysts

Catalysts suitable for use in conjunction with the presently disclosed matter can be catalysts capable of catalyzing RWGS reactions, i.e., hydrogenation of CO₂. In certain embodiments, the catalyst can be a solid catalyst, e.g., a solid-supported catalyst. The catalyst can be a metal oxide or mixed metal oxide. In certain embodiments, the catalyst can be located in a fixed packed bed, i.e., a catalyst fixed bed. In certain embodiments, the catalyst can include solid pellets, granules, plates, tablets, or rings. U.S. Patent Application Pub. No. 2013/0150466, the disclosure of which is hereby incorporated by reference in its entirety, describes additional catalysts that can be used in certain embodiments.

In certain embodiments, the catalyst can include one or more transition metals. The catalyst can include copper (Cu) or manganese (Mn). In certain embodiments, the catalyst can include both Cu and Mn. In certain embodiments, the catalyst can include Cu and Mn in a molar ratio of about 10:1 to about 1:10, about 4:1 to about 1:4, or about 1:1 (Cu:Mn). By way of non-limiting example, the molar ratio of Cu:Mn in the catalyst can be about 10:1, 9:1, 8:1, 7:1, 6:1, 5:1, 4:1, 3:1, 2.5:1, 2:1, 1.8:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.8, 1:2, 1:2.5, 1:3, 1:4, 1:5, 1:6, 1:7, 1:8, 1:9, or 1:10.

In certain embodiments, the catalyst can include a solid support. That is, the catalyst can be solid-supported. In certain embodiments, the solid support can include various metal salts, metalloid oxides, and/or metal oxides, e.g., titania (titanium oxide), zirconia (zirconium oxide), silica (silicon oxide), alumina (aluminum oxide), magnesia (magnesium oxide), and magnesium chloride. In certain embodiments, the solid support can include alumina (Al₂O₃), silica (SiO₂), magnesia (MgO), titania (TiO₂), zirconia (ZrO₂), cerium(IV) oxide (CeO₂), or a combination thereof. The amount of the solid support present in the catalyst can be between about 40% and about 95%, by weight, relative to the total weight of the catalyst. By way of non-limiting example, the solid support can constitute about 40%, 45%, 50%, 55%, 60%, 65%, 70%, 75%, 80%, 85%, 90%, or 95% of the total weight of the catalyst.

In certain embodiments, the catalyst can include one or more additional metals in addition to Cu and Mn. The additional metal(s) can include lanthanum (La), calcium (Ca), potassium (K), tungsten (W), and/or aluminum (Al). In certain embodiments, the additional metal(s) can be present in an amount between about 1% and 25%, relative to the total weight of the catalyst. For example, the catalyst can include about 1%, 2%, 3%, 4%, 5%, 6%, 7%, 8%, 9%, 10%, 11%, 12%, 13%, 14%, 15%, 16%, 18%, 20%, 22%, or 25% of the additional metal(s), by weight.

In certain embodiments, the catalyst can include about 1% to about 25% Cu, by weight. For example, the catalyst can include about 1%, 2%, 3%, 4%, 5%, 6%, 7%, 8%, 9%, 10%, 11%, 12%, 13%, 14%, 15%, 16%, 18%, 20%, 22%, or 25% Cu, by weight. In certain embodiments, the catalyst can include about 1% to about 25% Mn, by weight. For example, the catalyst can include about 1%, 2%, 3%, 4%, 5%, 6%, 7%, 8%, 9%, 10%, 11%, 12%, 13%, 14%, 15%, 16%, 18%, 20%, 22%, or 25% Mn, by weight. In certain embodiments, the catalyst can include about 10% Cu and about 10% Mn, by weight. By way of non-limiting example, the catalyst can include 10% Cu and 10% Mn, by weight. The remainder of the catalyst can be oxygen (i.e., the oxygen present in a metal oxide) and solid support (e.g., Al₂O₃).

Catalysts that include Cu and Mn can include Cu and Mn in various oxidation states. For example, Cu can be present in the catalyst as Cu(I) oxide (Cu₂O) and/or Cu(II) oxide (CuO). For example, Mn can be present in the catalyst as oxide (MnO). In certain embodiments, higher oxides of Mn initially present in the catalyst can be reduced in situ in the presence of H₂.

In certain embodiments, the catalysts of the presently disclosed subject matter can be free of chromium (Cr). In certain embodiments, the catalysts of the presently disclosed subject matter can contain low levels of Cr. The catalyst can include less than about 5% Cr, by weight. For example, the catalyst can include less than about 5%, 4%, 3%, 2%, 1%, 0.5%, 0.3%, 0.1%, 0.05%, 0.03%, 0.01%, 0.005%, or 0.001% Cr, by weight.

The catalysts of the presently disclosed subject matter can be prepared according to various techniques known in the art. For example, metal oxide catalysts suitable for use in RWGS reactions can be prepared from various metal nitrates, metal halides, metal salts of organic acids, metal hydroxides, metal carbonates, metal oxyhalides, metal sulfates, and the like. In certain embodiments, a transition metal oxide (e.g., a Cu or Mn oxide, or a mixed Cu/Mn oxide) can be precipitated along with a solid support (e.g., Al₂O₃). In certain embodiments, and as exemplified in the Examples below, catalysts can be prepared by precipitation of metal nitrates.

### Reaction Mixtures

The presently disclosed subject matter provides methods of converting mixtures of H₂ and CO₂ into syngas via the reverse water gas shift (RWGS) reaction. A mixture of H₂ and CO₂ can be termed a “reaction mixture.” The mixture of H₂ and CO₂ can alternatively be termed a “feed mixture” or “feed gas.”

The CO₂ in the reaction mixture can be derived from various sources. In certain embodiments, the CO₂ can be a waste product from an industrial process. In certain embodiments, CO₂ that remains unreacted in the RWGS reaction can be recovered and recycled back into the RWGS reaction.

Reaction mixtures suitable for use with the presently disclosed methods can include various proportions of H₂ and CO₂. In certain embodiments, the reaction mixture can include H₂ and CO₂ in a molar ratio (H₂:CO₂) between about 5:1 and about 1:2, e.g., about 5:1, 4:1, 3:1, 2.8:1, 2.6:1, 2.5:1, 2.4:1, 2.3:1, 2.2:1, 2.1:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9, or 1:2. The reaction mixture can include H₂ and CO₂ in a molar ratio (H₂:CO₂) of about 2:1 to about 1:1. In certain embodiments, the reaction mixture can include H₂ and CO₂ in a molar ratio (H₂:CO₂) of about 1.6:1.

### Methods of Preparing Syngas and Light Olefins

The methods of the presently disclosed subject matter include methods of preparing syngas. In one embodiment, an exemplary method can include providing a reaction chamber, as described above. The reaction chamber can include a solid-supported catalyst, as described above. The method can further include feeding a reaction mixture, as described above, to the reaction chamber. The method can additional include contacting H₂ and CO₂ (present in the reaction mixture) with the catalyst at a reaction temperature greater than 600° C., thereby inducing a RWGS reaction to provide a product mixture that includes H₂ and CO. The product mixture can further include H₂O (a product of the RWGS reaction, as shown in Equation 1) and unreacted CO₂.

The reaction mixture can be fed into the reaction chamber at various flow rates. The flow rate and gas hourly space velocity (GHSV) can be varied, as is known in the art. In certain embodiments, the GHSV can be about 200 h⁻¹ to about 5000 h⁻¹. For example, the GHSV can be about 370 h⁻¹ to about 400 h⁻¹.

The reaction temperature can be understood to be the temperature within the reaction chamber. The reaction temperature can influence the RWGS reaction, including conversion of CO₂ and H₂, the ratio of H₂:CO in the product mixture, and the overall yield. In certain embodiments, the reaction temperature can be greater than 560° C., e.g., greater than about 570° C., 580° C., 590° C., 600° C., 610° C., 620° C., 625° C., 630° C., 640° C., 650° C., 675° C., 700° C., 725° C., or 750° C. In certain embodiments, the reaction temperature can be greater than 600° C., e.g., greater than about 610° C., 620° C., 625° C., 630° C., 640° C., 650° C., 675° C., 700° C., 725° C., or 750° C. In certain embodiments, the reaction temperature can be between about 560° C. and about 800° C. In certain embodiments, the reaction temperature can be between about 600° C. and about 800° C. In certain embodiments, the reaction temperature can be about 670° C. In certain embodiments, the reaction temperature can be about 730° C.

The RWGS can proceed with partial conversion of CO₂ and H₂, thus providing a product mixture that includes CO, H₂O, CO₂, and H₂. In certain embodiments, the RWGS reaction can be performed to about 50% conversion of CO₂. Adjustment of the degree of conversion of CO₂ and H₂ as well as adjustment of the ratio of CO₂ and H₂ in the reaction mixture can therefore influence the ratio of H₂ and CO in the syngas product formed. For example, use of a higher molar ratio of H₂:CO₂ in the reaction mixture can increase the molar ratio of H₂:CO in the product mixture.

In certain embodiments, the product mixture can include H₂ and CO in a molar ratio (H₂:CO) of about 0.5:1 to about 5:1. In certain embodiments, the product mixture can include H₂ and CO in a molar ratio (H₂:CO) of about 1:1 to about 3:1, e.g., about 1:1, 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, or 3:1. In certain embodiments, the product mixture can include H₂ and CO in a molar ratio (H₂:CO) of about 1.5:1 to about 3:1, about 2:1 to about 3:1, or about 2.5:1. As noted above, the molar ratio (H₂:CO) of the product mixture can be influenced by the molar ratio (H₂:CO₂) of the reaction mixture.

In certain embodiments, the RWGS can be performed to relatively high conversion. That is, the amount of CO₂ present in the product mixture can be relatively low. In certain embodiments, the product mixture can include less than about 25% CO₂, by mole or less than about 20% CO₂, by mole. For example, the product mixture can include about 24%, 23%, 22%, 21%, 20%, 19%, 18%, 17%, 16%, 15%, 14%, 13%, 12%, 11%, 10%, 9%, 8% by mole.

In certain embodiments, the methods of the presently disclosed subject matter can include separating at least a portion of CO₂ and/or H₂O from the product mixture, to provide purified syngas. CO₂ and/or H₂O can be separated by various techniques known in the art. By way of non-limiting example, H₂O can be separated by condensation, e.g., by cooling the product mixture In certain embodiments, CO₂ can be removed from the product mixture and contributed to the reaction mixture, thereby recycling CO₂ through the RWGS reaction and improving overall economy of the process.

The presently disclosed subject matter also provides methods of preparing light olefins. In one embodiment, an exemplary method of preparing light olefins can include conducting a RWGS reaction to convert CO₂ and H₂ into a product mixture that includes H₂, CO, CO₂, and H₂O, as described above. The method can additionally include separating at least a portion of CO₂ and H₂O from the product mixture, to provide purified syngas. The method can further include subjecting purified syngas to a Fischer-Tropsch synthesis (FT) reaction to provide light olefins.

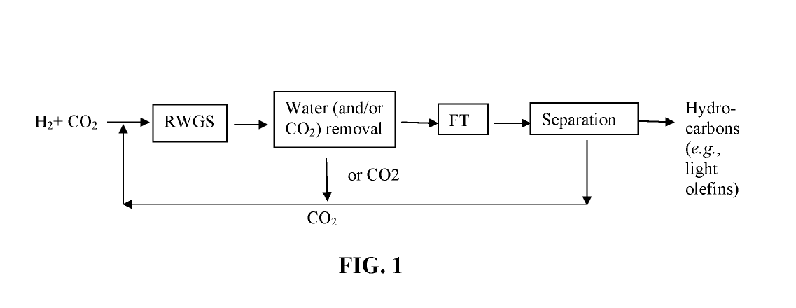

In certain embodiments, a RWGS reaction can be integrated with a FT reaction. As shown in FIG. 1, water can be removed from the product mixture obtained from a RWGS mixture to provide syngas, and the syngas can be fed into a FT reaction. CO₂ can optionally be separated from the product mixture from the RWGS reaction and/or the product mixture from the FT reaction and recycled back into the RWGS reaction. Separation of CO₂ from syngas obtained from the RWGS reaction before feeding the syngas into the FT reaction can increase olefin production capacity (measured per volume) of the FT reactor. In certain alternative embodiments, the product mixture from the RWGS reaction can be fed directly into the FT reaction without removal of CO₂. In certain embodiments, FT catalysts can tolerate the presence of CO₂, and CO₂ itself can participate in FT-type reactions.

The methods of the presently disclosed subject matter can have advantages over other techniques for preparation of syngas and preparation of light olefins. The presently disclosed subject matter includes the surprising discovery that catalysts containing Cu and/or Mn can be used to promote RWGS reactions at temperatures greater than 600° C. without sacrificing product purity or catalyst stability.

As noted above, the presently disclosed subject matter can involve use of catalysts that are free of Cr or contain low levels of Cr. It can be advantageous to avoid the use of Cr, as Cr can create environmental and handling concerns.

Additional advantages of the presently disclosed subject matter can include preparation of syngas with improved H₂:CO ratios. As demonstrated in the Examples, the methods of the presently disclosed subject matter can provide syngas containing H₂ and CO in a molar ratio of about 2:1 (e.g., 2.5:1), suitable for use in FT reactions. Moreover, the methods of the presently disclosed subject matter can prepare syngas via hydrogenation of CO₂ with minimal side reactions, good catalyst stability, good conversion of CO₂ (e.g., greater than 50%), and good yields of syngas. Additional advantages of the presently disclosed subject matter can include improved energy efficiency and overall economy.

For example, and without being bound to any particular theory, it can be that energy consumption for the RWGS reaction (hydrogenation of CO₂) is only about 10 kcal/mol, which is 5 times less than that of syngas generation by conventional methane steam reforming. In certain embodiments, preparation of syngas by the RWGS reaction could be coupled with preparation of syngas by conventional methane steam reforming to reduce the overall energy consumption of syngas preparation and improve overall energy efficiency.

### EXAMPLES

### Example 1

### Hydrogenation of CO₂ at 560° C. with a Catalyst Containing 10% Cu and 10% Mn

A reactor was charged with a catalyst containing 10% Cu and 10% Mn, by weight, supported on alumina. The catalyst was prepared according to the general procedure of Example 8. The catalyst loading was 0.84 g. The reactor was made of quartz, with a diameter of one inch and a length 45 cm. The reactor was located in a heated furnace. The temperature within the reactor was measured by a thermocouple located inside the reactor in special quartz tube thermos well with an inner diameter of about 6 mm. The thermocouple did not directly contact the gaseous components within the reactor. The reactor was heated to 560° C. A reaction mixture containing H₂ at a flow rate of 84 cc/min and CO₂ at a flow rate of 18.7 cc/min was fed into the reactor, thereby contacting the reaction mixture with the catalyst and inducing a RWGS reaction. The reaction mixture included H₂ and CO₂ in a molar ratio of about 4.5:1.

A product mixture containing H₂, CO₂, CO, and H₂O was removed from the reactor. Water was separated by condensation to provide a purified mixture. The purified mixture was then passed through a Genie Filter and then fed to a gas analyzer, where the composition of dry gas was determined. The composition of the purified mixture is presented in Table 1.

TABLE 1

| H₂ | 79.5% (by mole) |
|---|---|
| CO | 13.0% (by mole) |
| CO₂ | 7.5% (by mole) |
| H₂:CO | 6.1:1 (molar ratio) |
| conversion of CO₂ | 62.9% |

### Example 2

### Hydrogenation of CO₂ at 560° C. with a Catalyst Containing 10% Cu and 15% Mn

A reactor was charged with a catalyst containing 10% Cu and 15% Mn, by weight, supported on alumina. The catalyst was prepared according to the general procedure of Example 8, and the reactor was set up as in Example 1. The catalyst loading was 0.84 g. The reactor was heated to 560° C. A reaction mixture containing H₂ at a flow rate of 84 cc/min and CO₂ at a flow rate of 18.7 cc/min was fed into the reactor, thereby contacting the reaction mixture with the catalyst and inducing a RWGS reaction. The reaction mixture included H₂ and CO₂ in a molar ratio of about 4.5:1.

A product mixture containing H₂, CO₂, CO, and H₂O was removed from the reactor. Water was separated by condensation to provide a purified mixture. The purified mixture was then passed through a Genie Filter and then fed to a gas analyzer, where the composition of dry gas was determined. The composition of the purified mixture is presented in Table 2.

TABLE 2

| H₂ | 77.8% (by mole) |
|---|---|
| CO | 12.8% (by mole) |
| CO₂ | 7.4% (by mole) |
| H₂:CO | 6.07:1 (molar ratio) |
| conversion of CO₂ | 63.4% |

### Example 3

### Hydrogenation of CO₂ at 560° C. with a Catalyst Containing 5% Cu and 10% Mn

A reactor was charged with a catalyst containing 5% Cu and 10% Mn, by weight, supported on alumina. The catalyst was prepared according to the general procedure of Example 8, and the reactor was set up as in Example 1. The catalyst loading was 1.69 g. The reactor was heated to 560° C. A reaction mixture containing H₂ at a flow rate of 112 cc/min and CO₂ at a flow rate of 25 cc/min was fed into the reactor, thereby contacting the reaction mixture with the catalyst and inducing a RWGS reaction. The reaction mixture included H₂ and CO₂ in a molar ratio of about 4.5:1.

A product mixture containing H₂, CO₂, CO, and H₂O was removed from the reactor. Water was separated by condensation to provide a purified mixture. The purified mixture was then passed through a Genie Filter and then fed to a gas analyzer, where the composition of dry gas was determined. The composition of the purified mixture is presented in Table 3.

TABLE 3

| H₂ | 76.7% (by mole) |
|---|---|
| CO | 12.6% (by mole) |
| CO₂ | 7.3% (by mole) |
| H₂:CO | 6.08:1 (molar ratio) |
| conversion of CO₂ | 63.4% |

### Example 4

### Hydrogenation of CO₂ at 730° C. with a Catalyst Containing 10% Cu and 10% Mn and a Molar Ratio of H₂:CO₂ of About 4:1

A reactor was charged with a catalyst containing 10% Cu and 10% Mn, by weight, supported on alumina. The catalyst was prepared by pelletizing a precipitated dried gel of Cu, Mn, and Al, which followed the preparation described in Example 8. The reactor was set up as in Example 1. The catalyst loading was 8.4 g. The reactor was heated to 730° C. A reaction mixture containing H₂ at a flow rate of 87.2 cc/min and CO₂ at a flow rate of 21.8 cc/min was fed into the reactor, thereby contacting the reaction mixture with the catalyst and inducing a RWGS reaction. The reaction mixture included H₂ and CO₂ in a molar ratio of about 4:1.

A product mixture containing H₂, CO₂, CO, and H₂O was removed from the reactor. Water was separated by condensation to provide a purified mixture. The purified mixture was then passed through a Genie Filter and then fed to a gas analyzer, where the composition of dry gas was determined. The composition of the purified mixture is presented in Table 4.

TABLE 4

| H₂ | 78.5% (by mole) |
|---|---|
| CO | 16.1% (by mole) |
| CO₂ | 5.7% (by mole) |
| H₂:CO | 4.87:1 (molar ratio) |
| conversion of CO₂ | 73.5% |

### Example 5

### Hydrogenation of CO₂ at 730° C. with a Catalyst Containing 10% Cu and 10% Mn and a Molar Ratio of H₂:CO₂ of About 4:1

A reactor was charged with a pelletized catalyst containing 10% Cu and 10% Mn, by weight, impregnated on alumina. The catalyst was prepared according to the general procedure of Example 8, and the reactor was set up as in Example 1. The catalyst loading was 8.4 g. The reactor was heated to 730° C. A reaction mixture containing H₂ at a flow rate of 120 cc/min and CO₂ at a flow rate of 30 cc/min was fed into the reactor, thereby contacting the reaction mixture with the catalyst and inducing a RWGS reaction. The reaction mixture included H₂ and CO₂ in a molar ratio of about 4:1.

A product mixture containing H₂, CO₂, CO, and H₂O was removed from the reactor. Water was separated by condensation to provide a purified mixture. The purified mixture was then passed through a Genie Filter and then fed to a gas analyzer, where the composition of dry gas was determined. The composition of the purified mixture is presented in Table 5.

TABLE 5

| H₂ | 80.3% (by mole) |
|---|---|
| CO | 14.5% (by mole) |
| CO₂ | 5.1% (by mole) |
| H₂:CO | 5.5:1 (molar ratio) |
| conversion of CO₂ | 74.1% |

### Example 6

### Hydrogenation of CO₂ at 670° C. with a Catalyst Containing 10% Cu and 10% Mn and a Molar Ratio of H₂:CO₂ of About 2:1

A reactor was charged with a pelletized catalyst containing 10% Cu and 10% Mn, by weight, impregnated on alumina. The catalyst was prepared according to the general procedure of Example 8, and the reactor was set up as in Example 1. The catalyst loading was 8.4 g. The reactor was heated to 730° C. A reaction mixture containing H₂ at a flow rate of 32.8 cc/min and CO₂ at a flow rate of 16.4 cc/min was fed into the reactor, thereby contacting the reaction mixture with the catalyst and inducing a RWGS reaction. The reaction mixture included H₂ and CO₂ in a molar ratio of about 2:1.

A product mixture containing H₂, CO₂, CO, and H₂O was removed from the reactor. Water was separated by condensation to provide a purified mixture. The purified mixture was then passed through a Genie Filter and then fed to a gas analyzer, where the composition of dry gas was determined. The composition of the purified mixture is presented in Table 6.

TABLE 6

| H₂ | 62.0% (by mole) |
|---|---|
| CO | 21.47% (by mole) |
| CO₂ | 16.41% (by mole) |
| H₂:CO | 2.89:1 (molar ratio) |
| conversion of CO₂ | 56.3% |

### Example 7

### Hydrogenation of CO₂ at 670° C. with a Catalyst Containing 10% Cu and 10% Mn and a Molar Ratio of H₂:CO₂ of About 1.6:1

A reactor was charged with a pelletized catalyst containing 10% Cu and 10% Mn, by weight, impregnated on alumina. The catalyst was prepared according to the general procedure of Example 8, and the reactor was set up as in Example 1. The catalyst loading was 8.4 g. The reactor was heated to 670° C. A reaction mixture containing H₂ at a flow rate of 23.1 cc/min and CO₂ at a flow rate of 14.1 cc/min was fed into the reactor, thereby contacting the reaction mixture with the catalyst and inducing a RWGS reaction. The reaction mixture included H₂ and CO₂ in a molar ratio of about 1.6:1.

A product mixture containing H₂, CO₂, CO, and H₂O was removed from the reactor. Water was separated by condensation to provide a purified mixture. The purified mixture was then passed through a Genie Filter and then fed to a gas analyzer, where the composition of dry gas was determined. The composition of the purified mixture is presented in Table 7.

TABLE 7

| H₂ | 57.2% (by mole) |
|---|---|
| CO | 22.6% (by mole) |
| CO₂ | 20.14% (by mole) |
| H₂:CO | 2.5:1 (molar ratio) |
| conversion of CO₂ | 52.8% |

As demonstrated by Examples 1-7, use of a reaction temperature of about 670° C. and a reaction mixture containing H₂ and CO₂ in a molar ratio of about 1.6:1 in conjunction with a catalyst containing 10% Cu and 10% Mn, by weight, can have certain advantages. The RWGS reaction of Example 7 provided a product mixture containing H₂ and CO in a molar ratio of about 2.5:1. Syngas with a molar ratio of H₂: CO of about 2:1 (e.g., 2.5:1) can be useful for FT reactions, as noted above. Moreover, the RWGS reaction of Example provided good conversion of CO₂, at greater than 50% (52.8%).

### Example 8

### Preparation of a Catalyst Containing Cu and Mn

Nitrate salts of Cu, Mn, and Al—Cu(NO₃)₂, Mn(NO₃)₂, and Al(NO₃)₃— were dissolved in 200 mL of water. Ammonium hydroxide (NH₄OH) was then added dropwise until the pH of the solution was about 8, producing a metal oxide precipitate. The metal oxide precipitate was washed with distilled water and filtered. The filtered metal oxide was then dried for 12 hours at 120° C. and subsequently calcined for 8 hours at 650° C. The composition of the catalyst was then determined via conventional elemental analysis using X-ray fluorescence (XRF).

In one exemplary preparation, a catalyst supported on Al₂O₃ containing 10% Cu, by weight, and 10% Mn, by weight, was prepared by precipitating the quantities of nitrate salts presented in Table 8. The quantities of individual metal oxides present within the precipitated catalyst (as determined by XRF) are also presented in Table 8.

TABLE 8

| nitrate salts dissolved in 200 mL | | metal oxides | |
|---|---|---|---|
| water | | present in precipitate | |
| formula of nitrate | quantity of nitrate | formula | quantity of oxide |
| salt | salt (g) | of oxide | (g) |
| Mn(NO₃)₂•4H₂O | 7.03 | MnO | 1.99 |
| Cu(NO₃)₂•2.5H₂O | 5.63 | CuO | 1.93 |
| Al(NO₃)₃•9H₂O | 90.15 | Al₂O₃ | 12.26 |

### Example 9

### Evidence of Catalyst Stability

The catalysts used in Examples 1-7 were tested over more than 4 months. During this time, there was no observable change in catalyst activity, evidencing the stability of the catalysts under the reaction conditions.

Although the presently disclosed subject matter and its advantages have been described in detail, it should be understood that various changes, substitutions and alterations can be made herein without departing from the spirit and scope of the disclosed subject matter as defined by the appended claims. Moreover, the scope of the disclosed subject matter is not intended to be limited to the particular embodiments described in the specification. Accordingly, the appended claims are intended to include within their scope such alternatives.

1. A method of preparing syngas, comprising:
a. providing a reaction chamber that comprises a solid-supported catalyst comprising Cu and Mn;
b. feeding a reaction mixture comprising H₂ and CO₂ to the reaction chamber; and
c. contacting H₂ and CO₂ with the catalyst at a reaction temperature greater than 600° C. to provide a product mixture that comprises H₂ and CO.

2. The method of claim 1, wherein the catalyst comprises Cu and Mn in a molar ratio of about 4:1 to about 1:4.

3. The method of claim 2, wherein the catalyst comprises Cu and Mn in a molar ratio of about 1:1.

4. The method of claim 1, wherein the catalyst comprises one or more solid supports selected from the group consisting of Al₂O₃, MgO, SiO₂, TiO₂ and ZrO₂.

5. The method of claim 1, wherein the catalyst comprises one or more additional metals selected from the group consisting of La, Ca, K, W and Al.

6. The method of claim 5, wherein the catalyst comprises Al.

7. The method of claim 6, wherein the catalyst comprises about 10% Cu and about 10% Mn, by weight.

8. The method of claim 1, wherein the catalyst does not comprise Cr.

9. The method of claim 1, wherein the catalyst comprises less than about 1% Cr, by weight.

10. The method of claim 9, wherein the catalyst comprises less than about 0.1% Cr, by weight.

11. The method of claim 10, wherein the catalyst comprises less than about 0.01% Cr, by weight.

12. The method of claim 1, wherein the reaction mixture comprises H₂ and CO₂ in a molar ratio (H₂:CO₂) of about 1.6:1.

13. The method of claim 1, wherein the reaction temperature is greater than about 625° C., optionally greater than 650° C., or optionally greater than 670° C.

14. The method of claim 1, wherein the product mixture comprises H₂ and CO in a molar ratio (H₂:CO) of about 1:1 to about 3:1, optionally with a ratio of about 1.5:1 to about 3:1, or optionally with a ratio of about 2:1 to about 3:1, or optionally with a ratio of about 2.5:1.

15. The method of claim 1, wherein the product mixture further comprises CO₂ and H₂O.

16. The method of claim 15, wherein the product mixture comprises less than about 25% CO₂, by mole.

17. The method of claim 16, wherein the product mixture comprises less than about 20% CO₂, by mole.

18. The method of claim 17, further comprising separating at least a portion of CO₂ and H₂O from the product mixture to provide purified syngas.

19. A method of preparing light olefins, comprising:
a. providing a reaction chamber that comprises a solid-supported catalyst comprising Cu and Mn;
b. feeding a reaction mixture comprising H₂ and CO₂ to the reaction chamber; and
c. contacting H₂ and CO₂ with the catalyst at a reaction temperature greater than 600° C. to provide a product mixture that comprises H₂, CO, CO₂, and H₂O;
d. separating at least a portion of CO₂ and H₂O from the product mixture to provide purified syngas; and
e. subjecting purified syngas to a Fischer-Tropsch synthesis (FT) reaction to provide light olefins.
